(19) United States
(12) Patent Application Publication
(10) Pub. No.: US 2023/0415141 A1
(43) Pub. Date: Dec. 28, 2023

(54) LONG-LIFETIME SAPO-34 CATALYST PREPARED FROM MTO WASTE CATALYST AS RAW MATERIAL AND METHOD FOR PREPARATION THEREOF

(71) Applicant: China University of Petroleum (East China), Qingdao City (CN)

(72) Inventors: Xinmei LIU, Qingdao City (CN); Yue HU, Qingdao City (CN); Yuchao LYU, Qingdao City (CN); Jianye FU, Qingdao City (CN); Xuejie SUI, Qingdao City (CN); Zhengxing QIN, Qingdao City (CN)

(21) Appl. No.: 18/206,477

(22) Filed: Jun. 6, 2023

(30) Foreign Application Priority Data
Jun. 22, 2022 (CN) 202210828012.8

(51) Int. Cl. B01J 38/68; B01J 29/85; B01J 29/90; B01J 38/02; B01J 38/60; B01J 38/50; C01B 37/08

(52) U.S. Cl. CPC B01J 38/68; B01J 29/85; B01J 29/90; B01J 38/02; B01J 38/60; B01J 38/50; C01B 37/08; C01P 2002/01; C01P 2002/72

(57) ABSTRACT

The present invention provides a long-lifetime SAPO-34 catalyst prepared from waste MTO catalyst as a raw material and a preparation method thereof. The method comprises the following steps: mixing the waste MTO catalyst fine powder with water; adding a phosphoric acid and an organic amine and stirring to obtain an initial gel mixture for SAPO-34 molecular sieve; crystallizing the initial gel mixture and then at least drying it to obtain a raw SAPO-34 molecular sieve powder; calcining the raw molecular sieve powder to obtain a SAPO-34 molecular sieve powder; then mixing it with a binder and a matrix carrier in water with stirring, and then aging it; and molding and then calcining it to obtain the long-lifetime SAPO-34 catalyst. The preparation method of the present invention uses MTO waste catalyst as a raw material to synthesize SAPO-34 molecular sieve in situ within a short time, and to prepare MTO catalysts having a long life and high selectivity for light olefins.

### FIELD OF THE INVENTION

The present invention relates to a long-lifetime SAPO-34 catalyst prepared from a waste MTO catalyst as a raw material and a preparation method thereof, which belongs to the field of treatment and recycling of solid waste.

### BACKGROUND OF THE INVENTION

Among the SAPO-based catalysts, the SAPO-34 molecular sieve is particularly important. It is a microporous crystal composed of three tetrahedral structural units of SiO₂, AlO₂⁻, and PO²⁺. It has a unique CHA ellipsoidal cage and three-dimensional octahedral cross-pore channels with a pore size of about 0.38×0.38 nm². This typical microporous structure is the reason for its good shape selectivity. In 1990, the SAPO-34 molecular sieve, which shows excellent performance in methanol to olefin (MTO), was first reported and proved to have good regeneration and hydrothermal stability.

However, as a typical acidic catalyst, SAPO-34 molecular sieve has poor resistance to carbon deposition. The mechanical wear and sintering in the MTO reaction with high air velocity and strong exotherm for a long time can also lead to rapid deactivation of the SAPO-34 catalyst. Accordingly, the SAPO-34 molecular sieve must be regenerated by frequent carbon burning. After being used for a certain period, the catalyst can no longer meet the requirements of industrial production in terms of activity and crystal size. The catalyst eventually becomes the waste catalyst and is directly buried in a centralized landfill. However, as the waste catalysts mostly contain heavy aromatic hydrocarbons, their landfill disposal still has a high risk of environmental pollution and may easily lead to waste of resources.

Regarding a raw material for synthesis, boehmite, pseudo-boehmite, aluminum isopropoxide, or inorganic aluminum salts are usually used as aluminum sources. A 85 wt % phosphoric acid solution, solid ammonium phosphate or ammonium hydrogen phosphate are used as the phosphorus source. The silica sol, white carbon black or ethyl orthosilicate are mainly used as the silicon source. There is the problem of limited and expensive sources of a raw material. Therefore, the development of a method to synthesize long-lifetime SAPO-34 molecular sieves using waste catalysts is one of the pressing problems in the field.

### SUMMARY OF THE INVENTION

In view of above technical problems, it is an object of the present invention to provide a long-lifetime SAPO-34 catalyst prepared from MTO waste catalyst as a raw material and a preparation method thereof. The MTO waste catalysts are used as a raw material in the preparation method of the present invention and is able to synthesize SAPO-34 molecular sieves in situ and to prepare SAPO-34 catalysts with long life and high selectivity for light olefins.

To achieve the above object, the present invention first provides a method for preparing a long-lifetime SAPO-34 catalyst from a waste MTO catalyst as a raw material, which comprises the steps of:
(1) mixing the waste MTO catalyst fine powder with water and stirring them for a period of time to obtain a first mixture;
(2) adding phosphoric acid and an organic amine to the first mixture obtained in step (1) and stirring them for a period of time to obtain an initial gel mixture for SAPO-34 molecular sieve;
(3) crystallizing the initial gel mixture for SAPO-34 molecular sieve obtained in step (2) and then at least drying it to obtain a raw SAPO-34 molecular sieve powder;
(4) calcining the raw SAPO-34 molecular sieve powder obtained in step (3) to obtain a SAPO-34 molecular sieve powder;
(5) mixing the SAPO-34 molecular sieve powder obtained in step (4) with a binder and a matrix carrier in water with stirring, and then aging the resultant by standing it for a period of time to obtain a second mixture; and
(6) molding the second mixture obtained in step (5) and then calcining the resultant to obtain the long-lifetime SAPO-34 catalyst.

In the above preparation method, preferably, the fresh catalyst corresponding to the waste MTO catalyst fine powder used in step (1) is a SAPO-34 molecular sieve. The waste MTO catalyst fine powder has a Si/Al molar ratio of 1:(2-5), and more preferably 1:(3.5-4.5). More preferably, the waste MTO catalyst fine powder has a Si/Al/P molar ratio of 1:(2-5):(1-2.5). Particularly preferably, the Si/Al/P molar ratio of the waste MTO catalyst fine powder is 1:4:1.

In the above preparation method, preferably, the waste MTO catalyst fine powder used in step (1) is a permanently deactivated MTO catalyst, having no characteristic diffraction peaks of SAPO-34 molecular sieve in its X-ray diffraction spectrum. That is, in the X-ray diffraction spectra of the permanently deactivated catalyst fine powder, no characteristic diffraction peaks of the SAPO-34 framework is present at 9.6°, 12.8°, 16.2°, 21.5° and 30.9°. More preferably, the permanent deactivation of the waste MTO catalyst fine powder is achieved by exposing an incompletely deactivated waste MTO catalyst fine powder in air at room temperature for a long period of time (at least 3 months). The incompletely deactivated waste MTO catalyst fine powder is waste catalysts eliminated from industrial production.

The permanently deactivated waste MTO catalyst is inventively used in the present invention to synthesize SAPO-34 molecular sieve. The SAPO-34 framework in this permanently deactivated waste MTO catalyst collapsed, but a large number of SAPO-34 structural fragments such as microcrystalline structures or secondary structural units still exist. These are equivalent to providing a large number of nuclei during the crystallization process, leading to an increase in the supersaturated concentration of nuclei in the mother liquor, thus enabling the synthesis of small-crystal-size molecular sieves.

In the above preparation method, preferably, the waste MTO catalyst fine powder in step (1) can be calcined at a temperature of 500 to 750° C. for a time period of 4 to 12 hours.

In the above preparation method, preferably, in step (1), the first mixture is obtained by mixing the waste MTO catalyst fine powder with a certain amount of water and stirring them for 2 to 6 hours (for aging). More preferably, the stirring is carried out at a rotating speed of 400 to 700 r/min. This step can be carried out at room temperature.

In the above preparation method, preferably, the waste MTO catalyst fine powder is mixed with water in a mass ratio of 1:(5-50) in step (1), wherein, the water used in step (1) may be deionized water or distilled water.

In the above preparation method, preferably, in step (2), the initial gel mixture for SAPO-34 molecular sieve is obtained by adding phosphoric acid and an organic amine to the first mixture obtained in step (1) and stirring them for 2 to 4 hours (for aging). More preferably, the stirring is carried out at a rotating speed of 400 to 700 r/min. This step can be carried out at room temperature and the stirring is more preferably carried out at a temperature of 17 to 25° C.

In the above preparation method, preferably, in step (2), the organic amine comprises one or more of diethylamine, triethylamine, tetraethylammonium hydroxide, and morpholine.

In the above preparation method, preferably, in step (2), the phosphoric acid is in a form of an aqueous phosphoric acid solution. Preferably, in step (2), the phosphoric acid is in a form of a 85% (w/w) aqueous phosphoric acid solution.

In the above preparation method, preferably, the mass ratio of the waste MTO catalyst fine powder in step (1), the phosphoric acid and the organic amine in step (2) is 1:(0.2-1.5):(0.3-2.2). More preferably, the mass of phosphoric acid in this mass ratio is calculated in terms of the mass of a 85% (w/w) aqueous phosphoric acid solution. Particularly preferably, the mass ratio of waste MTO catalyst fine powder in step (1), the 85% (w/w) aqueous phosphoric acid solution and the organic amine in step (2) is 1:(0.2-0.6):(0.3-1.5).

In the above preparation method, preferably, the initial gel mixture for SAPO-34 molecular sieve obtained in step (2) has a pH of 5 to 10. More preferably, the initial gel mixture for SAPO-34 molecular sieve obtained in step (2) has a pH of 8 to 10. In step (2) of the present invention, a small amount of phosphoric acid is added to the mixture of waste MTO catalyst fine powder and water, which serves to adjust the pH of the system.

In the above preparation method, preferably, in step (3), the crystallization is carried out at a temperature of 160 to 220° C. for a time period of 5 to 48 hours. More preferably, the time period of the crystallization is 5 to 12 hours. More preferably, the crystallization is done by transferring the initial gel mixture for SAPO-34 molecular sieve into an autoclave for crystallization containing a PTFE liner and then placing the autoclave in an oven for crystallization. After the crystallization is completed, the crystallized product may be allowed to cool naturally to room temperature, followed by the subsequent steps of separation, washing and drying. The separation may be done by centrifugal separation to separate out the solid products. The washing may be done with deionized water, where the solid product obtained from the separation is washed until the pH of the liquid after washing is below 8. In addition, the order of separation and washing is not particularly limited in the present invention. It is possible to perform washing followed by separation, or to perform separation after each washing. These can be conventional operations in the art.

In the above preparation method, preferably, in step (3), the drying is carried out at a temperature of 100 to 120° C. for a time period of 4 to 12 hours.

In the above preparation method, preferably, in step (4), the calcining is carried out at a temperature of 500 to 600° C. for a time period of 4 to 10 hours. In the present invention, the raw SAPO-34 molecular sieve powder obtained from step (3) above is calcined at high temperature to remove the organic template, thereby obtaining the SAPO-34 molecular sieve powder.

In the above preparation method, preferably, in step (5), the binder comprises one or more of pseudo-boehmite, alumina sol, and silica sol.

In the above preparation method, preferably, in step (5), the matrix carrier comprises one or more of diatomaceous earth, kaolin, and montmorillonite.

In the above preparation method, preferably, in step (5), the SAPO-34 molecular sieve powder, binder, and matrix carrier are mixed in a mass ratio of 1:(0.1-1.25):(0.2-10). More preferably, in step (5), the SAPO-34 molecular sieve powder, binder, and matrix carrier are mixed in a mass ratio of 1:(0.1-0.8):(0.2-0.6).

In the above preparation method, preferably, in step (5), the SAPO-34 molecular sieve powder is mixed with water in a mass ratio of 1:(1-10). The water used may be deionized water or distilled water.

In the above preparation method, preferably, in step (5), the SAPO-34 molecular sieve powder obtained in step (4) is mixed with the binder and matrix carrier in water at a stirring speed of 400 to 700 r/min for a time period of 2 to 6 hours. This step may be carried out at room temperature.

In the above preparation method, preferably, in step (5), the aging by standing is carried out for a time period of 4 to 12 hours (more preferably 4 to 8 hours). The aging may be carried out at room temperature.

In the above preparation method, preferably, in step (6), the molding is carried out in a spray dryer with an inlet temperature of 250 to 350° C. and an outlet temperature of 80 to 200° C.

In the above preparation method, preferably, in step (6), the calcining is carried out at a temperature of 500 to 700° C. for a time period of 4 to 8 hours.

The present invention also provides a long-lifetime SAPO-34 catalyst prepared from a waste MTO catalyst as a raw material, obtained by the above-mentioned preparation method.

According to specific embodiments of the present invention, preferably, the long-lifetime SAPO-34 catalyst has an average crystal size of 200-600 nm.

According to specific embodiments of the present invention, preferably, the long-lifetime SAPO-34 catalyst has a mesoporous and macroporous structure with a mesopore size of 10-50 nm and a macroporous size of 50-200 nm.

The long-lifetime SAPO-34 catalyst of the present invention has a mesoporous structure and a hollow (i.e., macroporous) structure, which is caused by the etching of defective parts inside the synthesized crystal by the template in the mother liquor. Due to the ratio of each raw material as defined by the present invention and the permanently deactivated waste MTO catalyst fine powder used, the SAPO-34 crystals grow attached to smaller structural units. Meanwhile, the synthesis system of the present invention is an aluminum-rich system, i.e., the aluminum content is excessive, so the growth of the crystals is prone to forming the defects present in the form of end groups. These less ordered defective parts are easily etched by the mother liquor and preferentially dissolved. That is, the strongly alkaline organic amine as template will be slowly released into the mother liquor during the crystallization process, increasing the pH value of the mother liquor. Therefore, the structurally dense SAPO-34 molecular sieve, supposed to be well grown, is preferentially dissolved inside. With the extension of the crystallization time, the defective parts penetrate each other to form rich mesopores and hollow structures.

The hollow structure is present in the long-lifetime SAPO-34 catalyst of the present invention. The pore channels in the original microporous structure of SAPO-34 molecular sieve are easily blocked by accumulated carbon and deactivated rapidly, while the hollow structure can improve the volumetric carbon capacity of the molecular sieve. Therefore, more bulk accumulated carbon exists in the molecular sieve, thus extending its service life. Meanwhile, the presence of the hollow structure enhances the mass transfer ability of reactant molecules and product molecules, leading to more light olefins escaping from the pore channels, thus obtaining higher selectivity for light olefins.

The present invention also provides use of the long-lifetime SAPO-34 catalyst prepared from MTO waste catalyst as a raw material as described above in the reaction of methanol to light olefins (MTO).

Preferably, in the above use, the long-lifetime SAPO-34 catalyst achieves a total yield of 87% (wt %) or more of ethylene and propylene in the reaction of methanol to light olefins.

In the above use, preferably, the long-lifetime SAPO-34 catalyst described has a catalytic lifetime of 425-510 min in the reaction of methanol to light olefins.

The present invention provides a new idea of resource utilization of waste catalysts. It alleviates the pressure of solid waste disposal to a certain extent, and improves resource utilization. It also produces considerable economic benefits, and avoids the risk of environmental pollution caused by massive burial. The raw material for synthesis in the preparation method of the present invention is mainly the waste catalyst fine powder recovered from the three-stage cyclone separator in the MTO process equipment. It replaces the expensive industrial raw material such as inorganic or organic silicon source and aluminum source, while the amount of the template is small. This has advantages of reducing the production cost and simplifying the process flow, and also broadens the source of raw material for the synthesis of molecular sieve. Meanwhile, the preparation method of the present invention synthesizes SAPO-34 molecular sieves having high crystallinity without misoriented grains in a short time. Then, the preparation method of the present invention can synthesize the MTO fresh catalyst with long life and high selectivity for light olefins by adding and mixing a binder, a matrix carrier and other additives to the SAPO-34 molecular sieve having high crystallinity, and molding them by spraying, in a suitable way. The SAPO-34 molecular sieve catalyst of the present invention exhibits a long life of 510 min in the methanol to olefin reaction, and can achieve a high selectivity for light olefins (above 87%). It also can remain stable for a long time with high reaction stability, which is significantly better than the existing industrial catalysts and is well suited for industrial scale-up applications.

### BRIEF DESCRIPTION OF THE DRAWINGS

FIG. 1 shows a process flow diagram for preparing the long-lifetime SAPO-34 catalyst from a waste MTO catalyst as a raw material of Examples 1-5.

FIG. 2 shows X-ray diffraction spectra of the waste MTO catalyst, a fresh catalyst and catalyst samples of Examples 1-5.

FIG. 3a shows a transmission electron microscopy image of the long-lifetime SAPO-34 catalyst (S1) provided in Example 1.

FIG. 3b shows a transmission electron microscopy image of the long-lifetime SAPO-34 catalyst (S2) provided in Example 2.

FIG. 3c shows a transmission electron microscopy image of the long-lifetime SAPO-34 catalyst (S3) provided in Example 3.

FIG. 3d shows a transmission electron microscopy image of the long-lifetime SAPO-34 catalyst (S4) provided in Example 4.

### DETAILED DESCRIPTION OF THE INVENTION

The following detailed description of the technical solutions of the present invention is provided to have a clearer understanding of the technical features, objectives and beneficial effects of the present invention, but it is not to be understood as limiting the scope of the practicable scope of the present invention.

### Example 1

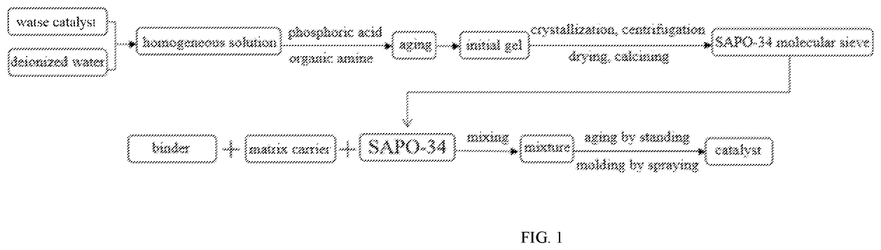

The example provides a long-lifetime SAPO-34 catalyst prepared from a waste MTO catalyst as a raw material, as in the process shown in FIG. 1, which is prepared by a method comprising the following steps:

8 g of waste MTO catalyst fine powders were mixed with 80 g of deionized water and stirred for 4 hours at room temperature for aging at a stirring speed of 400 to 700 r/min. Then 2.8 g of a 85% (w/w) aqueous phosphoric acid solution and 5.37 g of tetraethylammonium hydroxide were added sequentially and stirred for aging at room temperature for 2 hours at a stirring speed of 400 to 700 r/min to obtain an initial gel mixture. The initial gel mixture was loaded into an autoclave with a PTFE liner, and then the autoclave was placed in an oven at 160° C. for crystallization at a constant temperature for 8 hours. After crystallization, the autoclave was naturally cooled to room temperature, and then separated, washed, dried and calcined to obtain SAPO-34 molecular sieve powder. The separation was done by centrifugation to separate out the solid product; and the washing was done with deionized water to wash the separated solid product until the pH of the washed liquid was below 8. The drying was carried out at a temperature of 100° C. for a time period of 12 hours. The calcining was carried out at a temperature of 500° C. for a time period of 10 hours.

Then 10.5 g of the SAPO-34 molecular sieve powder were mixed with 6.5 g of alumina sol, 4.5 g of diatomaceous earth and 40 g of deionized water, stirred for 4 hours at room temperature at a stirring speed of 400 to 700 r/min to be well mixed, and then left standing to age for 4 hours to obtain a mixture. The mixture was then molded by spraying with a spray dryer with an inlet temperature of 350° C. and an outlet temperature of 180° C. The obtained product was calcined in a muffle furnace at 550° C. for 6 hours to obtain a long-lifetime SAPO-34 catalyst (Si).

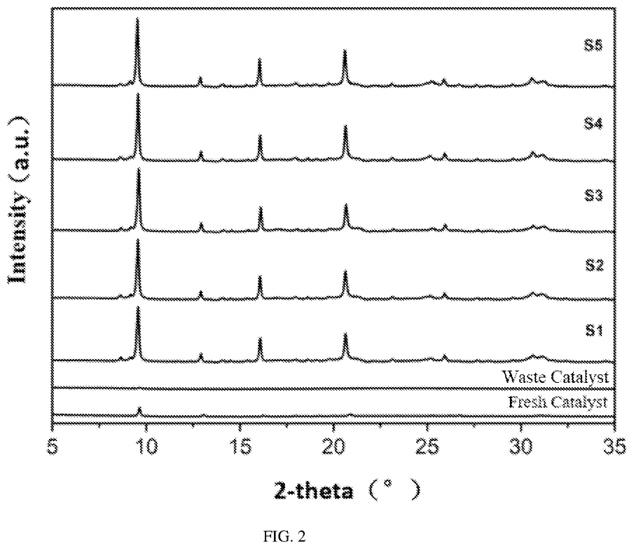

The XRD spectrum of the long-lifetime SAPO-34 catalyst (Si) is shown in FIG. 2. For comparison, the XRD spectra of the used waste MTO catalyst fine powders, an industrial fresh catalyst (which was the fresh one corresponding to the waste MTO catalyst fine powders used in this example) are also shown in FIG. 2. It can be demonstrated that the obtained samples are SAPO-34 molecular sieves with CHA topology, exhibiting strong characteristic diffraction peaks affiliated to the SAPO-34 framework at 9.6°, 12.8°, 16.2°, 21.5° and 30.9°. And its corresponding diffraction peak intensity was significantly higher than that of the industrial fresh catalyst, which can be used as a catalyst for the reaction of methanol to olefin.

Among them, the waste MTO catalyst fine powder was permanently deactivated waste MTO catalysts. As shown in FIG. 2, the characteristic diffraction peaks of SAPO-34 molecular sieve were not present in their X-ray diffraction spectra. Namely, the characteristic diffraction peaks of the SAPO-34 framework were not present at 9.6°, 12.8°, 16.2°, 21.5° and 30.9°. The permanent deactivation of the waste MTO catalyst fine powder in this example was achieved by exposing an incompletely deactivated waste MTO catalyst fine powder in air at room temperature for a long time period (3 months). This incompletely deactivated waste MTO catalyst fine powder was an industrially discarded waste MTO catalyst of SAPO-34 molecular sieve. The Si/Al/P molar ratio of the waste MTO catalyst fine powder used in this example was 1:(3.5-4.5):(1-2.5).

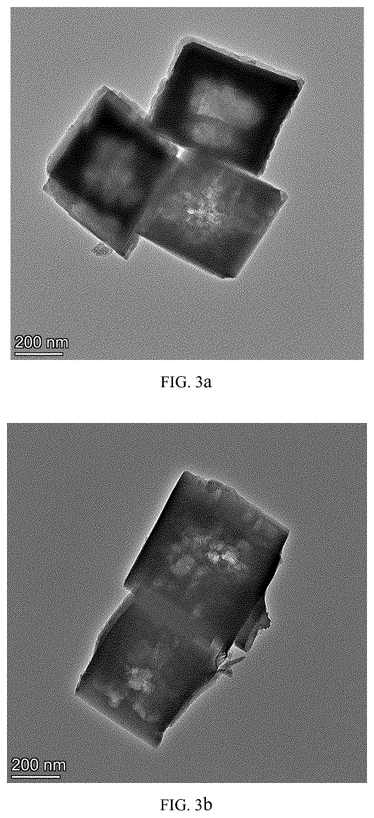

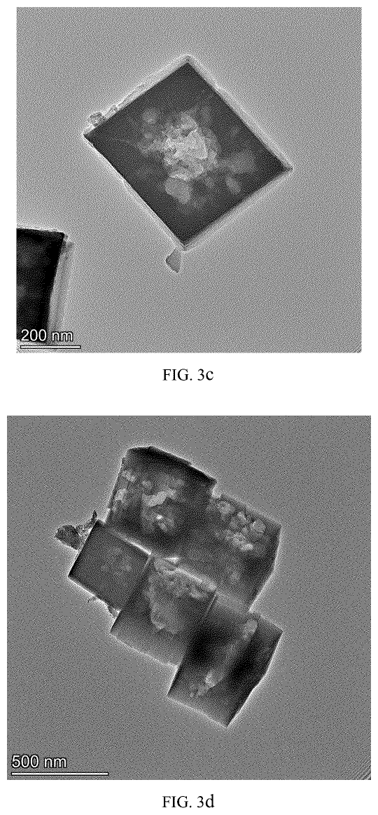

The transmission electron microscopy (TEM) image of the long-lifetime SAPO-34 catalyst (S1) is shown in FIG. 3a. The molecular sieve is cubic in shape with an average crystal size of 200 to 500 nm, and having a mesoporous and macropore structure with a mesopore size of 20 to 50 nm and a macropore size of 80 to 100 nm.

### Example 2

The example provides a long-lifetime SAPO-34 catalyst prepared from a waste MTO catalyst as a raw material, as in the process shown in FIG. 1, which is prepared by a method comprising the following steps:

g of waste MTO catalyst fine powders (the same as Example 1, which was a permanently deactivated waste MTO catalyst fine powder) were mixed with 120 g of deionized water and stirred for 2 hours at room temperature for aging at a stirring speed of 400 to 700 r/min. Then 6.5 g of a 85% (w/w) aqueous phosphoric acid solution and 13.65 g of diethylamine were added sequentially and stirred for aging at room temperature for 4 hours at a stirring speed of 400 to 700 r/min to obtain an initial gel mixture. The initial gel mixture was loaded into an autoclave with a PTFE liner, and then the autoclave was placed in an oven at 180° C. for crystallization at a constant temperature for 5 hours. After crystallization, the autoclave was naturally cooled to room temperature, and then separated, washed, dried and calcined to obtain SAPO-34 molecular sieve powder. The separation was done by centrifugation to separate out the solid product; and the washing was done with deionized water to wash the separated solid product until the pH of the washed liquid was below 8. The drying was carried out at a temperature of 110° C. for a time period of 6 hours. The calcining was carried out at a temperature of 550° C. for a time period of 5 hours.

Then 25 g of the SAPO-34 molecular sieve powder were mixed with 8.7 g of pseudo-boehmite, 6.3 g of kaolin and 40 g of deionized water, stirred for 2 hours at room temperature at a stirring speed of 400 to 700 r/min to be well mixed, and then left standing to age for 8 hours to obtain a mixture. The mixture was then molded by spraying with a spray dryer with an inlet temperature of 300° C. and an outlet temperature of 150° C. The obtained product was calcined in a muffle furnace at 550° C. for 8 hours to obtain a long-lifetime SAPO-34 catalyst (S2).

The XRD spectrum of the long-lifetime SAPO-34 catalyst (S2) is shown in FIG. 2. It can be demonstrated that the obtained samples are SAPO-34 molecular sieves with CHA topology, exhibiting strong characteristic diffraction peaks affiliated to the SAPO-34 framework at 9.6°, 12.8°, 16.2°, 21.5° and 30.9°. And its corresponding diffraction peak intensity was significantly higher than that of the industrial fresh catalyst, which can be used as a catalyst for the reaction of methanol to olefins.

The transmission electron microscopy (TEM) image of the long-lifetime SAPO-34 catalyst (S2) is shown in FIG. 3b. The molecular sieve is cubic in shape with an average crystal size of 200 to 500 nm, and having a mesoporous and macropore structure with a mesopore size of 10 to 30 nm and a macropore size of 50 to 70 nm.

### Example 3

The example provides a long-lifetime SAPO-34 catalyst prepared from a waste MTO catalyst as a raw material, as in the process shown in FIG. 1, which is prepared by a method comprising the following steps:

g of waste MTO catalyst fine powders (the same as Example 1, which was a permanently deactivated waste MTO catalyst fine powder) were mixed with 100 g of deionized water and stirred for 6 hours at room temperature for aging at a stirring speed of 400 to 700 r/min. Then 8 g of a 85% (w/w) aqueous phosphoric acid solution and 6.92 g of triethylamine were added sequentially and stirred for aging at room temperature for 3 hours at a stirring speed of 400 to 700 r/min to obtain an initial gel mixture. The initial gel mixture was loaded into an autoclave with a PTFE liner, and then the autoclave was placed in an oven at 220° C. for crystallization at a constant temperature for 12 hours. After crystallization, the autoclave was naturally cooled to room temperature, and then separated, washed, dried and calcined to obtain SAPO-34 molecular sieve powder. The separation was done by centrifugation to separate out the solid product; and the washing was done with deionized water to wash the separated solid product until the pH of the washed liquid was below 8. The drying was carried out at a temperature of 110° C. for a time period of 12 hours. The calcining was carried out at a temperature of 600° C. for a time period of 4 hours.

Then 19.5 g of the SAPO-34 molecular sieve powder were mixed with 8.7 g of pseudo-boehmite, 6.3 g of montmorillonite and 30 g of deionized water, stirred for 3 hours at room temperature at a stirring speed of 400 to 700 r/min to be well mixed, and then left standing to age for 6 hours to obtain a mixture. The mixture was then molded by spraying with a spray dryer with an inlet temperature of 300° C. and an outlet temperature of 150° C. The obtained product was calcined in a muffle furnace at 600° C. for 4 hours to obtain a long-lifetime SAPO-34 catalyst (S3).

The XRD spectrum of the long-lifetime SAPO-34 catalyst (S3) is shown in FIG. 2. It can be demonstrated that the obtained samples are SAPO-34 molecular sieves with CHA topology, exhibiting strong characteristic diffraction peaks affiliated to the SAPO-34 framework at 9.6°, 12.8°, 16.2°, 21.5° and 30.9°. And its corresponding diffraction peak intensity was significantly higher than that of the industrial fresh catalyst, which can be used as a catalyst for the reaction of methanol to olefin.

The transmission electron microscopy (TEM) image of the long-lifetime SAPO-34 catalyst (S3) is shown in FIG. 3c. The molecular sieve is cubic in shape with an average crystal size of 200 to 500 nm, and having a mesoporous and macropore structure with a mesopore size of 20 to 40 nm and a macropore size of 60 to 100 nm.

### Example 4

The example provides a long-lifetime SAPO-34 catalyst prepared from a waste MTO catalyst as a raw material, as in the process shown in FIG. 1, which is prepared by a method comprising the following steps:

The waste MTO catalyst fine powders (the same as Example 1, which was a permanently deactivated waste MTO catalyst fine powder) were calcined at a temperature of 600° C. for 8 hours. 5 g of calcined waste MTO catalyst fine powders were mixed with 60 g of deionized water and stirred for 6 hours at room temperature for aging at a stirring speed of 400 to 700 r/min. Then 2.4 g of a 85% (w/w) aqueous phosphoric acid solution and 7.2 g of morpholine were added sequentially and stirred for aging at room temperature for 3 hours at a stirring speed of 400 to 700 r/min to obtain an initial gel mixture. The initial gel mixture was loaded into an autoclave with a PTFE liner, and then the autoclave was placed in an oven at 175° C. for crystallization at a constant temperature for 24 hours. After crystallization, the autoclave was naturally cooled to room temperature, and then separated, washed, dried and calcined to obtain SAPO-34 molecular sieve powder. The separation was done by centrifugation to separate out the solid product; and the washing was done with deionized water to wash the separated solid product until the pH of the washed liquid was below 8. The drying was carried out at a temperature of 110° C. for a time period of 12 hours. The calcining was carried out at a temperature of 550° C. for a time period of 5 hours.

Then 9.5 g of the SAPO-34 molecular sieve powder were mixed with 5.1 g of silica sol, 2.8 g of kaolin and 20 g of deionized water, stirred for 4 hours at room temperature at a stirring speed of 400 to 700 r/min to be well mixed, and then left standing to age for 4 hours to obtain a mixture. The mixture was then spray formed with a spray dryer with an inlet temperature of 280° C. and an outlet temperature of 130° C. The obtained product was calcined in a muffle furnace at 500° C. for 8 hours to obtain a long-lifetime SAPO-34 catalyst (S4).

The XRD spectrum of the long-lifetime SAPO-34 catalyst (S4) is shown in FIG. 2. It can be demonstrated that the obtained samples are SAPO-34 molecular sieves with CHA topology, exhibiting strong characteristic diffraction peaks affiliated to the SAPO-34 framework at 9.6°, 12.8°, 16.2°, 21.5° and 30.9°. And its corresponding diffraction peak intensity was significantly higher than that of the industrial fresh catalyst, which can be used as a catalyst for the reaction of methanol to olefin.

The transmission electron microscopy (TEM) image of the long-lifetime SAPO-34 catalyst (S4) is shown in FIG. 3d. The molecular sieve is cubic in shape with an average crystal size of 200 to 400 nm, and having a mesoporous and macropore structure with a mesopore size of 10 to 40 nm and a macropore size of 60 to 80 nm.

### Example 5

The example provides a long-lifetime SAPO-34 catalyst prepared from a waste MTO catalyst as a raw material, as in the process shown in FIG. 1, which is prepared by a method comprising the following steps:

12 g of waste MTO catalyst fine powders (the same as Example 1, which was a permanently deactivated waste MTO catalyst fine powder) were mixed with 100 g of deionized water and stirred for 2 hours at room temperature for aging at a stirring speed of 400 to 700 r/min. Then 5.4 g of a 85% (w/w) aqueous phosphoric acid solution and 7.5 g of tetraethylammonium hydroxide were added sequentially and stirred for aging at room temperature for 3 hours at a stirring speed of 400 to 700 r/min to obtain an initial gel mixture. The initial gel mixture was loaded into an autoclave with a PTFE liner, and then the autoclave was placed in an oven at 200° C. for crystallization at a constant temperature for 48 hours. After crystallization, the autoclave was naturally cooled to room temperature, and then separated, washed, dried and calcined to obtain SAPO-34 molecular sieve powder. The separation was done by centrifugation to separate out the solid product; and the washing was done with deionized water to wash the separated solid product until the pH of the washed liquid was below 8. The drying was carried out at a temperature of 100° C. for a time period of 10 hours. The calcining was carried out at a temperature of 500° C. for a time period of 6 hours.

Then 16.5 g of the SAPO-34 molecular sieve powder were mixed with 3 g of pseudo-boehmite, 6.5 g of montmorillonite and 20 g of deionized water, stirred for 4 hours at room temperature at a stirring speed of 400 to 700 r/min to be well mixed, and then left standing to age for 6 hours to obtain a mixture. The mixture was then molded by spraying with a spray dryer with an inlet temperature of 350° C. and an outlet temperature of 180° C. The obtained product was calcined in a muffle furnace at 550° C. for 8 hours to obtain a long-lifetime SAPO-34 catalyst (S5).

The XRD spectrum of this long-lifetime SAPO-34 catalyst (S5) is shown in FIG. 2. It can be demonstrated that the obtained samples are SAPO-34 molecular sieves with CHA topology, exhibiting strong characteristic diffraction peaks affiliated to the SAPO-34 framework at 9.6°, 12.8°, 16.2°, 21.5° and 30.9°. And its corresponding diffraction peak intensity was significantly higher than that of the industrial fresh catalyst, which can be used as a catalyst for the reaction of methanol to olefins.

The evaluation on the performance of the molecular sieve catalyst Five samples of the long-lifetime SAPO-34 catalyst from Examples 1-5 were evaluated for MTO performance using a fixed-bed catalytic reaction device for evaluation. First, the above five catalyst samples and 1.0 g of industrial fresh catalyst (which was the fresh catalyst corresponding to the waste MTO catalyst fine powder used in Examples 1-5) were weighed and placed into the reactor and activated by nitrogen gas at 550° C. for 2 hours, and then cooled down to 470° C. The feed methanol was carried by nitrogen at an air speed of 1.5 h⁻¹ and the reaction products were analyzed online using gas chromatography Agilent 6820 and Agilent 7820 at 15 min intervals. The reaction was terminated when the methanol conversion rate was below 100%, i.e., when methanol and dimethyl ether components appeared in the GC Agilent 6820 spectrum. After the reaction, the liquid products were collected in an ice and water bath and the gaseous products were discharged through the tail gas duct. The evaluation results are shown in Table 1.

TABLE 1

| | Life | Selectivity (wt %) | | | | | | | | |
|---|---|---|---|---|---|---|---|---|---|---|
| Samples | (min) | CH₄ | C₂H₆ | C₂H₄ | C₃H₈ | C₃H₆ | C₄ | C₅⁺ | C_{2═} + C_{3═} | C_{2═} + C_{3═} + C_{4═} |
| Fresh | 50 | 5.0 | 51.7 | 0.5 | 32.9 | 0.4 | 8.7 | 0.8 | 84.6 | 93.3 |
| catalyst | | | | | | | | | | |
| S1 | 510 | 4.8 | 57.2 | 0.6 | 29.9 | 0.5 | 6.8 | 0.2 | 87.1 | 93.1 |
| S2 | 485 | 3.5 | 57.5 | 0.6 | 30.4 | 1.2 | 5.9 | 0.9 | 87.9 | 93.7 |
| S3 | 425 | 4.3 | 59.1 | 0.7 | 28.8 | 1 | 5.2 | 0.9 | 87.9 | 93 |
| S4 | 440 | 4.6 | 58.2 | 0.6 | 29.5 | 0.5 | 6.2 | 0.4 | 87.7 | 93.8 |
| S5 | 470 | 4.6 | 59.1 | 0.5 | 29.4 | 0.5 | 5.4 | 0.5 | 88.5 | 93.9 |

As can be seen from Table 1, all five catalyst samples of the inventive examples had a long catalytic lifetime (higher than 425 min), which was more than 8 times that of the industrial fresh catalyst. Meanwhile the total yield of ethylene and propylene could exceed 87%.

What is claimed is:

1. A method for preparing a long-lifetime SAPO-34 catalyst from waste MTO catalyst as a raw material, comprising the steps of:
(1) mixing the waste MTO catalyst fine powder with water and stirring them for a period of time to obtain a first mixture;
(2) adding phosphoric acid and an organic amine to the first mixture obtained in step (1) and stirring them for a period of time to obtain an initial gel mixture for SAPO-34 molecular sieve;
(3) crystallizing the initial gel mixture for SAPO-34 molecular sieve obtained in step (2) and then at least drying it to obtain rawSAPO-34 molecular sieve powder;
(4) calcining the raw SAPO-34 molecular sieve powder obtained in step (3) to obtain SAPO-34 molecular sieve powder;
(5) mixing the SAPO-34 molecular sieve powder obtained in step (4) with a binder and a matrix carrier in water with stirring, and then aging the resultant by standing it for a period of time to obtain a second mixture; and
(6) molding the second mixture obtained in step (5) and then calcining the resultant to obtain the long-lifetime SAPO-34 catalyst.

2. The method according to claim 1, wherein the fresh catalyst corresponding to the waste MTO catalyst fine powder used in step (1) is a SAPO-34 molecular sieve; and
the waste MTO catalyst fine powder used in step (1) is a permanently deactivated waste MTO catalyst, having no characteristic diffraction peaks of SAPO-34 molecular sieve in its X-ray diffraction spectrum.

3. The method according to claim 1, wherein in step (1), the first mixture is obtained by mixing the waste MTO catalyst fine powder with a certain amount of water and stirring them for 2 to 6 hours.

4. The method according to claim 1, wherein in step (1) the waste MTO catalyst fine powder is mixed with water in a mass ratio of 1:(5-50).

5. The method according to claim 1, wherein in step (2), the initial gel mixture for SAPO-34 molecular sieve is obtained by adding phosphoric acid and an organic amine to the first mixture obtained in step (1) and stirring them for 2 to 4 hours.

6. The method according to claim 1, wherein in step (2), the organic amine comprises one or more of diethylamine, triethylamine, tetraethylammonium hydroxide, and morpholine.

7. The method according to claim 1, wherein in step (2), the phosphoric acid is in a form of an aqueous phosphoric acid solution; preferably, in step (2), the phosphoric acid is in a form of a 85% (w/w) aqueous phosphoric acid solution.

8. The method according to claim 1, wherein the mass ratio of the waste MTO catalyst fine powder in step (1), the phosphoric acid and the organic amine in step (2) is 1:(0.2-1.5):(0.3-2.2).

9. The method according to claim 1, wherein the initial gel mixture for SAPO-34 molecular sieve obtained in step (2) has a pH of 5 to 10.

10. The method according to claim 1, wherein in step (3), the crystallization is carried out at a temperature of 160 to 220° C. for a time period of 5 to 48 hours.

11. The method according to claim 1, wherein in step (3), the drying is carried out at a temperature of 100 to 120° C. for a time period of 4 to 12 hours.

12. The method according to claim 1, wherein in step (4), the calcining is carried out at a temperature of 500 to 600° C. for a time period of 4 to 10 hours.

13. The method according to claim 1, wherein in step (5), the aging by standing is carried out for a time period of 4 to 12 hours.

14. The method according to claim 1, wherein in step (5), the binder comprises one or more of pseudo-boehmite, alumina sol, and silica sol.

15. The method according to claim 1, wherein in step (5), the matrix carrier comprises one or more of diatomaceous earth, kaolin, and montmorillonite.

16. The method according to claim 1, wherein in step (5), the SAPO-34 molecular sieve powder, the binder, and the matrix carrier are mixed in a mass ratio of 1:(0.1-1.25):(0.2-10).

17. The method according to claim 1, wherein in step (5), the SAPO-34 molecular sieve powder is mixed with water in a mass ratio of 1:(1-10).

18. The method according to claim 1, wherein in step (6), the molding is carried out in a spray dryer with an inlet temperature of 250 to 350° C. and an outlet temperature of 80 to 200° C.

19. The method according to claim 1, wherein in step (6), the calcining is carried out at a temperature of 500 to 700° C. for a time period of 4 to 8 hours.

20. A long-lifetime SAPO-34 catalyst prepared from waste MTO catalyst as a raw material by the method according to claim 1.
